(19) United States
(12) Patent Application Publication
(10) Pub. No.: US 2025/0296988 A1
(43) Pub. Date: Sep. 25, 2025

(54) COMBINATION THERAPY

(71) Applicant: ViiV Healthcare Company, Wilmington, DE (US)

(72) Inventors: ZHI HONG, Research Triangle Park, NC (US); Jerry Lee JEFFREY, Research Triangle Park, NC (US); Brian Alvin JOHNS, Research Triangle Park, PA (US); Andrew SPALTENSTEIN, Raleigh, NC (US); Emile Johann VELTHUISEN, Research Triangle Park, NC (US)

(21) Appl. No.: 18/255,920

(22) PCT Filed: Dec. 3, 2021
(86) PCT No.: PCT/US2021/061697

§ 371 Date: Jun. 5, 2023

Related U.S. Application Data
(60) Provisional application No. 63/122,031, filed on Dec. 7, 2020.

(51) Int. Cl. C07K 16/10; A61K 31/4985; A61K 39/00; A61P 31/18

(52) U.S. Cl. CPC C07K 16/1063; A61K 31/4985; A61P 31/18; A61K 2039/505

(57) ABSTRACT

The present invention relates to a combination of cabotegravir or a pharmaceutically acceptable salt thereof and a gp120 binding protein. The present invention also provides a method of treatment human immunodeficiency virus (HIV) with the co-administration of a therapeutically effective amount of cabotegravir or a pharmaceutically acceptable salt thereof and a therapeutically effective amount of a gp120 binding protein.

### CROSS REFERENCE TO RELATED APPLICATIONS

The instant application claims priority to U.S. Provisional Patent Application Ser. No. 63/122,031 filed Dec. 7, 2020. The content of this application is incorporated by reference herein in its entirety.

### SEQUENCE LISTING

The instant application contains a Sequence Listing, which has been submitted electronically in computer readable form in .txt format and is hereby incorporated by reference in its entirety. Said .txt file, created on Jun. 5, 2023, is named PU66989_US_Seqlist_created_20May2021.txt and is 27,738 bytes.

### FIELD OF THE INVENTION

The invention relates to a method of treatment of Human Immunodeficiency Virus (HIV) infection. In particular, the invention relates to injectable combinations for the treatment of HIV infection.

### BACKGROUND TO THE INVENTION

Human Immunodeficiency Virus type 1 (HIV-1) infection, and the resulting Acquired Immunodeficiency Syndrome (AIDS), remain threats to global public health, despite extensive efforts to develop anti-HIV-1 therapeutic agents. HIV-1 possesses a high mutation rate and a high frequency of recombination, which can result in rapid emergence of drug-resistant variants when the viral replication is not sufficiently inhibited. Iyidogan, P., & Anderson, K. S. (2014). Current perspectives on HIV-1 antiretroviral drug resistance. Viruses, 6 (10), 4095-4139. Problems with compliance to administration regimens, poor tolerability and previous exposure to antiretroviral therapy also place a patient in danger of developing HIV-1 drug resistant strains. Obasa A E, Mikasi S G, Brado D, et al. Front Microbiol. 2020; 11:438, Rossouw T M, Feucht U D, Melikian G, et al. PLOS One. 2015; 10 (7).

Highly active antiretroviral therapy (HAART), focuses on the co-administration of different drugs that inhibit viral replication by several mechanisms, specifically the HIV replication enzymes protease, integrase and transaminase. Inhibition of multiple mechanisms is necessary because propagation of the virus with resistance to a single agent becomes inhibited by the action of the other two agents. Shafer R W, Vuitton D A. Biomed Pharmacother. 1999 March; 53 (2): 73-86. Unfortunately, HIV drug resistance reduces or even eliminates the efficacy of multiple mechanism inhibition.

New strategies in the continued fight against HIV-1 drug resistance include antiretroviral therapies that focus on long acting formulations to reduce the number of dosage administrations. Another treatment strategy to avoid drug resistance includes neutralizing HIV-1 with broadly neutralizing antibodies (bNAbs). Neutralization is defined as the loss of infectivity that occurs when an antibody molecule binds a virion, the complete infective form of the virus outside a host cell. Specific bNAbs bind to HIV-1 envelope CD4-binding site of the virus membrane glycoprotein gp120, neutralizing the virion's ability to attach to and pass its RNA to T cells. Neutralizing antibodies have been developed that identify HIV-1 with varying recognition and sufficient areas of glycoprotein amino acid sequence conservation. Burton, D., Mascola, J. Nat Immunol 16, 571-576 (2015).

Currently, there remains a need in the art to develop HIV-1 therapeutic regimens that treat infection by increasing compliance to administration through fewer dosages.

### SUMMARY OF THE INVENTION

According to a first aspect of the invention, there is provided a combination comprising cabotegravir or a pharmaceutically acceptable salt thereof and a gp120 binding protein wherein the gp120 binding protein neutralizes HIV-1.

According to a second aspect of the invention, there is provided a method of treating HIV in a human in need thereof comprising co-administering to a human a therapeutically effective amount of cabotegravir or a pharmaceutically acceptable salt thereof and a therapeutically effective amount of a gp120 binding protein wherein the gp120 binding protein neutralizes HIV-1.

According to a third aspect of the invention, there is provided a combination comprising cabotegravir or a pharmaceutically acceptable salt thereof and a gp120 binding protein wherein the gp120 binding protein neutralizes HIV-1 for the use in treatment of HIV.

According to a further aspect of the invention, there is provided use of cabotegravir or a pharmaceutically acceptable salt thereof and a gp120 binding protein as defined by claims 1 to 13, in the manufacture or a medicament for use in the treatment of HIV.

In a final aspect of the invention, there is provided a kit comprising cabotegravir or a pharmaceutically acceptable salt thereof and a gp120 binding protein that neutralizes HIV-1.

The present invention is advantageous in a number of respects. Specifically, the method of co-administration of cabotegravir or a pharmaceutically acceptable salt thereof and a gp120 binding protein may be safe, stable over an extended period of time and effective to treat HIV. A combination according to the invention comprising cabotegravir or a pharmaceutically acceptable salt thereof cabotegravir and a gp120 binding protein may provide protection against HIV infection and HIV neutralization.

### DETAILED DESCRIPTION OF THE INVENTION

### Definitions

As used herein, the term ‘pharmaceutical composition’ means a composition that is suitable for pharmaceutical use.

As used herein, the term “combination” refers to at least two therapeutic agents to be co-administered. As used herein the term “therapeutic agent” is understood to mean a substance that produces a desired effect in a tissue, system, animal, mammal, human, or other subject. For example, non-fixed dose combinations are contemplated.

As used herein, the term “co-administer” refers to simultaneous or sequential administration such that therapeutically effective amounts of the compounds are both present in the body of the patient. The term “co-administer” also refers to administration at the same time, as part of a non-fixed dose optionally in more than one formulation. The term “co-administer” also refers to administration at different schedules but could be administered within a given treatment cycle. Co-administration includes administration of pharmaceutical composition of integrase strand transfer inhibitors and gp120 binding protein, for example, administration of cabotegravir or a pharmaceutically acceptable salt thereof and a gp120 binding protein within seconds, minutes, hours, days or weeks of the administration of one another. For example, in some embodiments, a unit dose of one of the integrase strand transfer inhibitor or the gp120 binding protein is administered first, followed within seconds or minutes by administration of the other, by either the same or different routes.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html).

Suitable pharmaceutically acceptable salts can include acid or base addition salts Suitable pharmaceutically acceptable salts of the invention include base addition salts.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

“Therapeutically effective amount” or “effective amount” refers to that amount of the compound being administered that will prevent a condition or will relieve to some extent one or more of the symptoms of the disorder being treated. Pharmaceutical compositions suitable for use herein include compositions wherein the active ingredients are contained in an amount sufficient enough to achieve the intended purpose. Determination of a therapeutically effective amount is well within the capability of those skilled in the art, especially in light of the detailed disclosure provided herein.

As used herein, the term “treatment” or “treating” in the context of therapeutic methods, refers to alleviating the specified condition, eliminating or reducing the symptoms of the condition, slowing or eliminating the progression, invasion, or spread of the condition and reducing or delaying the reoccurrence of the condition in a previously afflicted subject. The present invention further provides use of the compounds or compositions of the invention for the preparation of a medicament for the treatment of several conditions in a mammal (e.g., human) in need thereof.

As used herein, the term “parenteral” or “parenterally” in the context of therapeutic methods, refers to a route of administration of a pharmaceutical compound or composition other than by oral administration. Parenteral routes of administration suitable for use herein include injection, infusion, implantation or some other route other than the alimentary canal. Parenteral routes of injection administration include intravenous, intramuscular and subcutaneous.

As used herein, the term “gp120 binding protein” refers to antibodies and other protein constructs, such as domains, that are capable of binding to envelope glycoprotein GP120. The terms “gp120 binding protein” and “antigen binding protein” are used interchangeably herein. This does not include the natural cognate ligand or receptor. An example of a gp120 binding protein is the antibody N6 disclosed in U.S. Pat. No. 10,562,960 having the complementarity determining regions (CDR) SEQ ID Nos: 1, 2, 15, 16, 17, 18, 19 or 20. N6LS is a gp120 binding protein 960 having the complementarity determining regions (CDR) SEQ ID Nos: 1, 2, 15, 16, 17, 18, 19 or 20, and an IgG1 constant domain comprising M428L and N434S mutations.

As used herein, the term “antibody” is used herein in the broadest sense to refer to molecules with an immunoglobulin-like domain (for example IgG, IgM, IgA, IgD or IgE) and includes monoclonal or fragment thereof, recombinant, polyclonal, chimeric, human, humanised, broadly neutralizing, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or Δ light chain. The majority of antibodies in the serum belong to the IgG class, there are four isotypes of human IgG (IgG1, IgG2, IgG3 and IgG4), the sequences of which differ mainly in their hinge region.

Fully human antibodies can be obtained using a variety of methods, for example using yeast-based libraries or transgenic animals (e.g. mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

As used herein, the term “antigen binding site” refers to a site on an antigen binding protein that is capable of specifically binding to an antigen, this may be a single variable domain, or it may be paired VH/VL domains as can be found on a standard antibody. Single-chain Fv (ScFv) domains can also provide antigen-binding sites.

As used herein, the term “multi-specific antibody” refers to an antibody that comprises at least two different antigen binding sites. Each of these antigen-binding sites is capable of binding to a different epitope, which may be present on the same antigen or different antigens. The multi-specific antigen binding protein may have specificity for more than one antigen, for example two antigens, or three antigens, or four antigens.

Classification and formats of bispecific antibodies are comprehensively described in reviews by Labrijn et al 2019 and Brinkmann and Kontermann 2017. Bispecifics may be generally classified as having a symmetric or asymmetric architecture. Bispecifics may have an Fc or may be fragment-based (lacking an Fc). Fragment based bispecifics combine multiple antigen-binding antibody fragments in one molecule without an Fc region e.g. Fab-scFv, Fab-scFv2, orthogonal Fab-Fab, Fab-Fv, tandem scFc (e.g. BiTE and BIKE molecules), Diabody, DART, TandAb, scDiabody, tandem dAb etc.

Symmetric formats combine multiple binding specificities in a single polypeptide chain or single HL pair including Fc-fusion proteins of fragment-based formats and formats whereby antibody fragments are fused to regular antibody molecules. Examples of symmetric formats may include DVD-Ig, TVD-Ig, CODV-Ig, (scFv) 4-Fc, IgG-(scFv) 2, Tetravalent DART-Fc, F (ab) 4CrossMab, IgG-HC-scFv, IgG-LC-scFv, mAb-dAb etc.

Asymmetric formats retain as closely as possible the native architecture of natural antibodies by forcing correct HL chain pairing and/or promoting H chain heterodimerization during the co-expression of three (if common heavy or light chains are used) or four polypeptide chains e.g. Triomab, asymmetric reengineering technology immunoglobulin (ART-Ig), CrossMab, Biclonics common light chain, ZW1 common light chain, DuoBody and knobs into holes (KiH), DuetMab, κλ body, Xmab, YBODY, HET-mAb, HET-Fab, DART-Fc, SEEDbody, mouse/rat chimeric IgG.

Bispecific formats also include an antibody fused to a non-Ig scaffold such as Affimabs, Fynomabs, Zybodies, and Anticalin-IgG fusions, ImmTAC.

As used herein, the term “chimeric antigen receptor” (“CAR”) as used herein, refers to an engineered receptor that consists of an extracellular antigen binding domain (usually derived from a monoclonal antibody or fragment thereof, e.g. a VH domain and a VL domain in the form of a scFv), optionally a spacer region, a transmembrane region, and one or more intracellular effector domains. CARs have also been referred to as chimeric T cell receptors or chimeric immunoreceptors (CIRs). CARs are genetically introduced into hematopoietic cells, such as T cells, to redirect T cell specificity for a desired cell-surface antigen, resulting in a CAR-T therapeutic.

The term “spacer region” as used herein, refers to an oligo- or polypeptide that functions to link the transmembrane domain to the target binding domain. This region may also be referred to as a “hinge region” or “stalk region”. The size of the spacer can be varied depending on the position of the target epitope in order to maintain a set distance (e.g. 14 nm) upon CAR: target binding.

The term “transmembrane domain” as used herein, refers to the part of the CAR molecule that traverses the cell membrane.

The term “intracellular effector domain” (also referred to as the “signalling domain”) as used herein refers to the domain in the CAR that is responsible for intracellular signalling following the binding of the antigen binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines.

It will be appreciated by a person skilled in the art that VH and/or VL domains disclosed herein may be incorporated, e.g. in the form of a scFv, into CAR-T therapeutics.

As used herein, the term “neutralises” as used throughout the present specification means that the biological activity of gp120 is reduced in the presence of an antigen binding protein as described herein in comparison to the activity gp120 in the absence of the antigen binding protein, in vitro or in vivo. Neutralisation may be due to one or more of blocking gp120 binding to its receptor, preventing gp120 from activating its receptor, down regulating gp120 or its receptor, or affecting effector functionality.

As used herein, the term “CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342:877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1
Kabat Numbering Scheme

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

Accordingly, an antigen binding protein is provided, which comprises any one or a combination of the following CDR amino acid sequences:

CDRH1 of SEQ ID NO 15:
AHILF
CDRH2 of SEQ ID NO 16:
WIKPQYGAVNFGGGFRD
CDRH3 of SEQ ID NO 17:
DRSYGDSSWALDA
CDRL1 of SEQ ID NO 18:
QTSQGVGSDLH
CDRL2 of SEQ ID NO 19:
HTSSVED
CDRL3 of SEQ ID NO 20:
QVLQF

In an embodiment of the invention, CDRs may be modified by at least one amino acid substitution, deletion or addition, wherein the variant antigen binding protein substantially retains the biological characteristics of the unmodified protein, such as N6 or N6LS.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 2 below.

TABLE 2
conservative substitutions

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the flanking residues that comprise the CDR as part of alternative definition(s) e.g. Kabat or Chothia, may be substituted with a conservative amino acid residue.

Such antigen binding proteins comprising variant CDRs as described above may be referred to herein as “functional CDR variants”.

As used herein, the term “epitope” as used herein refers to that portion of the antigen that makes contact with a particular binding domain of the antigen binding protein, also known as the paratope. An epitope may be linear or conformational/discontinuous. A conformational or discontinuous epitope comprises amino acid residues that are separated by other sequences, i.e. not in a continuous sequence in the antigen's primary sequence assembled by tertiary folding of the polypeptide chain. Although the residues may be from different regions of the polypeptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. Epitope mapping can be carried out using various techniques known to persons skilled in the art as described in publications such as Methods in Molecular Biology ‘Epitope Mapping Protocols’, Mike Schutkowski and Ulrich Reineke (volume 524, 2009) and Johan Rockberg and Johan Nilvebrant (volume 1785, 2018). Exemplary methods include peptide based approaches such as pepscan whereby a series of overlapping peptides are screened for binding using techniques such as ELISA or by in vitro display of large libraries of peptides or protein mutants, e.g. on phage. Detailed epitope information can be determined by structural techniques including X-ray crystallography, solution nuclear magnetic resonance (NMR) spectroscopy and cryogenic-electron microscopy (cryo-EM). Mutagenesis, such as alanine scanning, is an effective approach whereby loss of binding analysis is used for epitope mapping. Another method is hydrogen/deuterium exchange (HDX) combined with proteolysis and liquid-chromatography mass spectrometry (LC-MS) analysis to characterize discontinuous or conformational epitopes.

Competition between the gp120 binding protein of the invention and a reference gp120 binding protein, e.g. a reference antibody, may be determined by one or more techniques known to the skilled person such as ELISA, FMAT, Surface Plasmon Resonance (SPR) or FORTEBIO OCTET Bio-Layer Interferometry (BLI). Such techniques may also be referred to as epitope binning. There are several possible reasons for this competition: the two proteins may bind to the same or overlapping epitopes, there may be steric inhibition of binding, or binding of the first protein may induce a conformational change in the antigen that prevents or reduces binding of the second protein.

The reduction or inhibition in biological activity may be partial or total. In an embodiment of the invention, administration of a therapeutically effective amount of a disclosed antibody or antigen binding fragment that binds to gpl20 can reduce or inhibit an HIV-1 infection (for example, as measured by infection of cells, or by number or percentage of subjects infected by HIV-1, or by an increase in the survival time of infected subjects) by a desired amount, for example by at least 10%, at least 20%, at least 50%, at least 60%, at least 70%, at least 80%, at least 90%, at least 95%, at least 98%, or even at least 100% (elimination or prevention of detectable HIV-1 infection), as compared to a suitable control. Neutralisation may be determined or measured using one or more assays known to the skilled person or as described herein.

“Percent identity” between a query nucleic acid sequence and a subject nucleic acid sequence is the “Identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN, FASTA, ClustalW, MUSCLE, MAFFT, EMBOSS Needle, T-Coffee, and DNASTAR Lasergene. Importantly, a query nucleic acid sequence may be described by a nucleic acid sequence identified in one or more claims herein.

“Percent identity” between a query amino acid sequence and a subject amino acid sequence is the “Identities” or “Identical” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact.

The variant sequence substantially retains the biological characteristics of the unmodified protein, such as gp120.

For sequence variation, the VH or VL (or HC or LC) sequence may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s).

The HC sequence may be a variant sequence with up to 20% sequence variation.

The LC sequence may be a variant sequence with up to 20% sequence variation.

The sequence variation may exclude one or more or all of the CDRs, for example the CDRs are the same as the VH or VL (or HC or LC) sequence and the variation is in the remaining portion of the VH or VL (or HC or LC) sequence, so that the CDR sequences are fixed and intact.

Typically, the variation is a substitution, particularly a conservative substitution, for example as shown in Table 2.

The variant sequence substantially retains the biological characteristics of the unmodified protein, such as gp120.

As used herein, the term “genetic barrier to resistance” refers to the number of mutations required to confer resistance to a pharmaceutical composition. A pharmaceutical composition with a low genetic barrier to resistance will become less effective after the virus mutates or have a low number of mutations. A pharmaceutical composition with a high genetic barrier to resistance will not lose biological activity against the virus even after several mutations.

As used herein, the term “integrase strand transfer inhibitor” refers to a class of antiretroviral drugs designed to block the action of integrase, a viral enzyme that inserts the viral genome into the DNA of the host cell. While not being bound by theory, inhibition of HIV genome integration stops retroviral replication halting the further spread of the virus.

### STATEMENT OF THE INVENTION

The present invention, a combination and a method, may be used to treat or neutralize HIV which unless further clarified is intended to mean HIV-1. As an alternative embodiment, the method and combination may also be effective against HIV-2, or against patients having dual HIV-1/HIV-2 infection.

According to a first aspect, the present invention provides a combination comprising cabotegravir or a pharmaceutically acceptable salt thereof and a gp120 binding protein wherein the gp120 binding protein neutralizes HIV-1.

Cabotegravir (N-((2,4-Difluorophenyl)methyl)-6-hydroxy-3-methyl-5,7-dioxo-2,3,5,7,11,11a hexahydro (1,3) oxazolo (3,2-a)pyrido (1,2-d) pyrazine-8-carboxamide) is described in U.S. Pat. No. 8,129,385 in example Z-1. Cabotegravir is an integrase strand transfer inhibitor (INSTI) that exhibits subnanomolar potency and antiviral activity against a broad range of HIV-1 strains. Oral administration of Cabotegravir has exhibited acceptable safety and tolerability profiles, a long half-life, and few drug-drug interactions. Cabotegravir has been demonstrated to be efficacious in treatment and prevention of HIV both in oral and parenteral dosage forms, see for instance, Margolis D A, Brinson C C, Eron J J, et al. 744 and Rilpivirine as Two Drug Oral Maintenance Therapy: LAI116482 (LATTE) Week 48 Results. 21st Conference on Retroviruses and Opportunistic Infections (CROI); Mar. 3-6, 2014; Boston, MA, Margolis D A, Podzamczer D, Stellbrink H-J, et al. Cabotegravir+Rilpivirine as Long-Acting Maintenance Therapy: LATTE-2 Week 48 Results. 21st International AIDS Conference; Jul. 18-22, 2016; Durban, South Africa, Abstract THAB0206 LB. Levin: Conference reports for National AIDS Treatment Advocacy Project (NATAP); 2016, and Markowitz M, Frank I, Grant R, et al. ECLAIR: Phase 2A Safety and PK Study of Cabotegravir LA in HIV-Uninfected Men. Abstract presented at 23rd Conference on Retroviruses and Opportunistic Infections (CROI); Feb. 22-25, 2016; Boston, MA. Bictegravir is also an integrase strand transfer inhibitor. Herein disclosed is a combination of bictegravir with a gp120 binding molecule.

In an embodiment of the invention, cabotegravir is present as a free acid. In an alternative embodiment of the invention, Cabotegravir is present as a sodium salt.

In an embodiment of the invention, cabotegravir or pharmaceutically acceptable salt thereof is presented as a pharmaceutical composition. The present cabotegravir or pharmaceutically acceptable salt thereof can be administered orally or parenterally. In the case of oral administration, the cabotegravir or pharmaceutically acceptable salt thereof can be also used as a conventional preparation, for example, as any dosage form of a solid agent such as tablets, powders, granules, capsules and the like; an aqueous agent; an oily suspension; or a liquid agent such as syrup and elixir.

In the case of parenteral administration, the compound can be used as an aqueous or oily suspension injectable, or a nasal drop. Upon preparation of it, conventional excipients, binders, lubricants, aqueous solvents, oily solvents, emulsifiers, suspending agents, preservatives, stabilizers and the like may be used.

In an embodiment of the invention, the pharmaceutical composition comprises cabotegravir or pharmaceutically acceptable salt thereof and a surfactant system. In an embodiment of the invention, the surfactant system further comprises a combination of polymers providing for the release of cabotegravir or pharmaceutically acceptable salt thereof over a period of one to three months. A suitable combination of polymers is, for example, polysorbate 20 and polyethylene glycol 3350. In an embodiment of the invention, the surfactant system comprises mannitol. In an embodiment of the invention, the cabotegravir is nanomilled to <200 nm.

In an embodiment of the invention, the dose of cabotegravir or pharmaceutically acceptable salt thereof to be administered orally is be calculated on a basis of about 1 mg/day to about 50 mg/day, preferably 5 mg/day to about 30 mg/day, more preferably 30 mg/day.

In another embodiment of the invention, the dose of cabotegravir or pharmaceutically acceptable salt thereof is to be administered parenterally at a dose of about 10 mg to about 1000 mg per week, per one month, per two months, per three months, or per six months. According to one embodiment, the compound or salt thereof is administered at either 400 mg, 600 mg, or 900 mg.

In an embodiment of the invention, the pharmaceutical composition as defined above may be administered daily, weekly, monthly, bi-monthly (once every two months) or tri-monthly (once every three months)

In an embodiment of the invention, the combination provides a gp120 binding protein. GP120 binding proteins bind to the exposed envelope glycoprotein 120 found on the HIV virion and inhibit the virion from attaching to CD4 receptor found on the cell surface of T cells. By blocking binding to the CD4 receptor, the virion is unable to bind with T cell coreceptors, fuse the virion membrane with the T cell membrane and push the virion's genetic information into the cell.

In an embodiment of the invention, the gp120 binding protein comprises a monoclonal antibody or a fragment thereof. The term “monoclonal antibody” indicates the character of the antibody as being obtained from a substantially homogeneous population of antibodies and is not to be construed as requiring production of the antibody by any particular method. In an embodiment of the invention, the monoclonal antibody has a VH and VL as defined above.

In an embodiment of the invention, the gp120 binding protein comprises a heavy chain variable region (VH) comprising a heavy chain complementarity determining region (CDRH) having a CDRH1 amino acid sequence that comprises a sequence that is at least 80%, 85%, 90%, 95%, 98% or 99% identical to SEQ ID NO: 15, a CDRH2 amino acid sequence that comprises a sequence that is at least 80%, 85%, 90%, 95%, 98% or 99% identical to SEQ ID NO: 16, and a CDRH3 amino acid sequence that comprises a sequence that is at least 80%, 85%, 90%, 95%, 98% or 99% identical to SEQ ID NO: 17, and a light chain variable region (VL) comprising a light chain complementarity determining region (CDRL) having a CDRL1 amino acid that comprises a sequence that is at least 80%, 85%, 90%, 95%, 98% or 99% identical to SEQ ID NO: 18, a CDRL2 amino acid sequence that comprises a sequence that is at least 80%, 85%, 90%, 95%, 98% or 99% identical to SEQ ID NO: 19, and a CDRHL 3 amino acid sequence that comprises a sequence that is at least 80%, 85%, 90%, 95%, 98% or 99% identical to SEQ ID NO: 20.

In an embodiment of the invention, the gp120 binding protein comprises a VH comprising a heavy chain complementarity determining region (CDRH) having the CDRH1 amino acid sequence as shown in SEQ ID NO: 15, the CDRH2 amino acid sequence as shown in SEQ ID NO: 16, and the CDRH3 amino acid sequence as shown in SEQ ID NO: 17, and a VL comprising a light chain complementarity determining region (CDRL) having the CDRL1 amino acid sequence as shown in SEQ ID NO: 18, the CDRHL amino acid sequence as shown in SEQ ID NO: 19, and the CDRHL 3 amino acid sequence shown in in SEQ ID NO: 20.

In an embodiment of the invention, the gp120 binding protein comprises comprise a variable heavy chain region amino acid sequence that comprises a sequence that is at least 80%, 85%, 90%, 95%, 98%, or 99% identical to SEQ ID NO: 1. In an embodiment of the invention, the gp120 binding protein comprises a variable heavy chain region amino acid sequence as shown in SEQ ID NO: 1.

In an embodiment of the invention, the gp120 binding protein comprises comprise a variable light chain region amino acid sequence that comprises a sequence that is at least 80%, 85%, 90%, 95%, 98%, or 99% identical to SEQ ID NO: 2. In an embodiment of the invention, the gp120 binding protein comprises a variable heavy chain region amino acid sequence as shown in SEQ ID NO: 2.

In an embodiment of the invention, the gp120 binding protein comprises comprise a variable heavy chain region amino acid sequence that comprises a sequence that is at least 80%, 85%, 90%, 95%, 98%, or 99% identical to SEQ ID NO: 1 and a variable light chain region amino acid sequence that comprises a sequence that is at least 80%, 85%, 90%, 95%, 98%, or 99% identical to SEQ ID NO: 2. In an embodiment of the invention, the gp120 binding protein comprises a variable heavy chain region amino acid sequence as shown in SEQ ID NO: 1 and a variable heavy chain region amino acid sequence as shown in SEQ ID NO: 2.

In an embodiment of the invention, the gp120 binding protein may have a CDRH1 with the amino acid sequence at least 80%, 85%, 90%, 95%, 98%, or 99% identical to SEQ ID NO: 15 and a CDRH2 with the amino acid sequence at least 80%, 85%, 90%, 95%, 98%, or 99% identical to SEQ ID NO: 16. In an embodiment of the invention, the gp120 binding protein may have a CDRH1 with the amino acid sequence at least 80%, 85%, 90%, 95%, 98%, or 99% identical to SEQ ID NO: 15 and a CDRH3 with the amino acid sequence at least 80%, 85%, 90%, 95%, 98%, or 99% identical to SEQ ID NO: 17. In an embodiment of the invention, the gp120 binding protein may have a CDRH2 with the amino acid sequence at least 80%, 85%, 90%, 95%, 98%, or 99% identical to SEQ ID NO: 16 and a CDRH3 with the amino acid sequence at least 80%, 85%, 90%, 95%, 98%, or 99% identical to SEQ ID NO: 17.

In an embodiment of the invention, the gp120 binding protein may have a CDRH1 with the amino acid sequence of SEQ ID NO: 15 and a CDRH2 with the amino acid sequence of SEQ ID NO: 16. In an embodiment of the invention, the gp120 binding protein may have a CDRH1 with the amino acid sequence of SEQ ID NO: 15 and a CDRH3 with the amino acid sequence of SEQ ID NO: 17. In an embodiment of the invention, the gp120 binding protein may have a CDRH2 with the amino acid sequence of SEQ ID NO: 16 and a CDRH3 with the amino acid sequence of SEQ ID NO: 17.

In an embodiment of the invention, the gp120 binding protein may have a CDRL1 with the amino acid sequence at least 80%, 85%, 90%, 95%, 98%, or 99% identical to SEQ ID NO: 18 and a CDRL2 with the amino acid sequence at least 80%, 85%, 90%, 95%, 98%, or 99% identical to SEQ ID NO: 19. In an embodiment of the invention, the gp120 binding protein may have a CDRL1 with the amino acid sequence at least 80%, 85%, 90%, 95%, 98%, or 99% identical to SEQ ID NO: 18 and a CDRL3 with the amino acid sequence at least 80%, 85%, 90%, 95%, 98%, or 99% identical to SEQ ID NO: 20. In an embodiment of the invention, the gp120 binding protein may have a CDRL2 with the amino acid sequence at least 80%, 85%, 90%, 95%, 98%, or 99% identical to SEQ ID NO: 19 and a CDRL3 with the amino acid sequence at least 80%, 85%, 90%, 95%, 98%, or 99% identical to SEQ ID NO: 20.

In an embodiment of the invention, the gp120 binding protein may have a CDRL1 with the amino acid sequence of SEQ ID NO: 18 and a CDRL2 with the amino acid sequence of SEQ ID NO: 19. In an embodiment of the invention, the gp120 binding protein may have a CDRL1 with the amino acid sequence of SEQ ID NO: 18 and a CDRL3 with the amino acid sequence of SEQ ID NO: 20. In an embodiment of the invention, the gp120 binding protein may have a CDRL2 with the amino acid sequence of SEQ ID NO: 19 and a CDRL3 with the amino acid sequence of SEQ ID NO: 20.

In an embodiment of the invention, the gp120 binding protein in an antigen binding fragment. In an embodiment of the invention, the antigen binding fragment has a VH and a VL as defined above. In an embodiment of the invention, the antigen binding fragment comprises a Fv, Fab, F(ab′)₂, scFV or a scFV₂ fragment. Further examples of antigen binding fragments include but are not limited to Fv, Fab, Fab′, Fab′-SH, F(ab′)2, diabodies, linear antibodies, single-chain antibody molecules (e.g. scFV) and multispecific antibodies formed from antibody fragments.

In an embodiment of the invention, the constant region of the antibody includes one or more amino acid substitutions to optimize in vivo half-life of the antibody. The serum half-life of IgG Abs is regulated by the neonatal Fc receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g., Hinton et al, J Immunol, 176:346-356, 2006); M428L and N434S (the “LS” mutation, see, e.g., Zalevsky, et al, Nature Biotechnology, 28:157-159, 2010); N434A (see, e.g., Petkova et al, Int. Immunol, 18:1759-1769, 2006); T307A, E380A, and N434A (see, e.g., Petkova et al, Int. Immunol, 18:1759-1769, 2006); and M252Y, S254T, and T256E (see, e.g., Dall'Acqua et al, J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions. Thus, in an embodiment of the invention, the monoclonal antibody comprises a recombinant constant domain comprising a modification that increases binding to a neonatal Fc receptor relative to an unmodified constant domain, wherein the recombinant domain is an IgG1 constant domain comprising M428L and N434S mutations.

In an embodiment of the invention, the constant region of the antibody includes one of more amino acid substitutions to optimize antibody-dependent cell-mediated cytotoxicity (ADCC). ADCC is mediated primarily through a set of closely related Fey receptors. In some embodiments, the antibody includes one or more amino acid substitutions that increase binding to FcγRIIIa. Several such substitutions are known to the person of ordinary skill in the art, such as substitutions at IgG constant regions S239D and I332E (see, e.g., Lazar et al, Proc. Natl, Acad. Sci. U.S.A., 103:4005-4010, 2006); and S239D, A330L, and I332E (see, e.g., Lazar et al, Proc. Natl, Acad. Sci. U.S.A., 103:4005-4010, 2006).

In an embodiment of the invention, combinations of the above substitutions are also included, to generate an IgG constant region with increased binding to FcRn and FcγRIIIa. The combinations increase antibody half-life and ADCC. For example, such combination include antibodies with the following amino acid substitution in the Fc region: (1) S239D/1332E and T250Q/M428L; (2) S239D/1332E and M428L/N434S; (3) S239D/I332E and N434A; (4) S239D/I332E and T307A/E380A/N434A; (5) S239D/1332E and M252Y/S254T/T256E; (6) S239D/A330L/1332E and 250Q/M428L; (7) S239D/A330L/1332E and M428L/N434S; (8) S239D/A330L/1332E and N434A; (9) S239D/A330L/1332E and T307A/E380A/N434A; or (10) S239D/A330L/1332E and M252Y/S254T/T256E. In some examples, the antibodies, or an antigen binding fragment thereof is modified such that it is directly cytotoxic to infected cells, or uses natural defenses such as complement, antibody dependent cellular cytotoxicity (ADCC), or phagocytosis by macrophages.

In an embodiment of the invention, the gp120 binding protein is provided as a gp120 binding protein composition. In an embodiment, the gp120 binding protein composition includes one or more of the gp120-specific antibody, antigen binding fragment, conjugate, CAR, T cell expressing a CAR, or nucleic acid molecule encoding such molecules, that are disclosed herein in a carrier. The gp120 binding protein compositions are useful, for example, for the treatment or detection of HIV-1 infection. The gp120 binding protein compositions can be prepared in unit dosage forms for administration to a subject. The amount and timing of administration are at the discretion of the treating physician to achieve the desired purposes. The gp120-specific antibody, antigen binding fragment, conjugate, CAR, T cell expressing a CAR, or nucleic acid molecule encoding such molecules can be formulated for systemic or local administration. In one example, the gp120-specific antibody, antigen binding fragment, conjugate, CAR, T cell expressing a CAR, or nucleic acid molecule encoding such molecules, is formulated for parenteral administration, such as intravenous administration.

In an embodiment of the invention, the gp120 binding protein compositions comprise an antibody, antigen binding fragment, or conjugate thereof, in at least 70% (such as at least 75%, at least 80%, at least 85%, at least 90%, at least 95%, at least 96%, at least 97%, at least 98% or at least 99% purity. In certain embodiments, the compositions contain less than 10% (such as less than 5%, less than 4%, less than 3%, less than 2%, less than 1%, less than 0.5%, or even less) of macromolecular contaminants, such as other mammalian (e.g., human) proteins

The gp120 binding protein compositions for administration can include a solution of the gpl20-specific antibody, antigen binding fragment, conjugate, CAR, T cell expressing a CAR, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely and will be selected primarily based on fluid volumes, viscosities, body weight and the like in accordance with the particular mode of administration selected and the subject's needs.

A typical gp120 binding protein composition for intravenous administration includes about 0.01 to about 30 mg/kg of antibody or antigen binding fragment or conjugate per subject per day (or the corresponding dose of a conjugate including the antibody or antigen binding fragment). Actual methods for preparing administrable compositions will be known or apparent to those skilled in the art and are described in more detail in such publications as Remington's Pharmaceutical Science, 22nd ed., Pharmaceutical Press, London, UK (2012). In some embodiments, the composition can be a liquid formulation including one or more antibodies, antigen binding fragments (such as an antibody or antigen binding fragment that specifically binds to gpl20), in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, or an antigen binding fragment thereof or a conjugate or a nucleic acid encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and typically administered at a dosage of 0.5 to 15 mg/kg of body weight. Considerable experience is available in the art in the administration of antibody drugs, which have been marketed in the U.S. since the approval of RITUXAN® in 1997. Antibodies, antigen binding fragments, conjugates, or a nucleic acid encoding such molecules, can be administered by slow infusion, rather than in an intravenous push or bolus.

In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

In an embodiment of the invention, the gp120 binding protein or gp120 binding protein composition, as defined above, is administered at a dose in the range of from about 1, 1.25, 1.5, 2, 2.5, 3, 3.5, 4, 4.5, 5, 5.5, 6, 6.5, 7, 7.5, 8, 8.5, 9, 9.5 or 10 mg/kg, or other dose deemed appropriate by the treating physician. In an embodiment of the invention, the gp120 binding protein or gp120 binding protein composition can be administered to a subject at a dose of from about 0.5 to about 40 mg/kg, such as about 1 to about 30, about 1 to about 20, about 1 to about 15, about 1 to about 10, about 1 to about 5, about 1 to about 3, about 0.5 to about 40 mg/kg, such as about 0.5 to about 30, about 0.5 to about 20, about 0.5 to about 15, about 0.5 to about 10, about 0.5 to about 5, about 0.5 to about 3, about 3 to about 7, about 8 to about 12, about 15 to about 25, about 18 to about 22, about 28 to about 32, about 10 to about 20, about 5 to about 15, or about 20 to about 40 mg/kg. The doses described herein can be administered according to the dosing frequency/frequency of administration described herein, including without limitation daily, weekly, every 2 weeks, monthly, every other month, every 3 months, every 4 months, every 5 months, every 6 months, every 9 months, or every 12 months.

In an embodiment of the invention, the combination is be administered by any suitable means. In one embodiment, the combination is administered parenterally. The combination may be administered in the form of an injectable. In an embodiment of the invention, the combination is administered intramuscularly. In an embodiment of the invention, the combination is administered subcutaneously. In an embodiment of the invention, the combination is administered intravenously.

In an embodiment of the invention, the combination is administered to a human once every month, once every 2 months or once every 3 months. In an embodiment of the invention, the combination is administered once every month. In an embodiment of the invention, the combination is administered once every 2 months. In an embodiment of the invention, the combination is administered once every 3 months. In an embodiment of the invention, the combination is administered once every 6 months.

In an embodiment of the invention, the cabotegravir and the gp120 binding protein are self-administered by a human. The term “self-administered”, as used herein, means administration by someone other than a healthcare professional, for example, a patient may administer the pharmaceutical composition to themselves, or someone else, other than a healthcare professional may administer the pharmaceutical composition to the patient. In another embodiment, the cabotegravir and the gp120 binding protein are administered by a health-care professional.

In an embodiment of the invention, the gp120 binding protein is a broadly neutralizing antibody (bNAb). Broadly neutralizing antibodies to HIV-1 are distinct from other antibodies to HIV-1 in that they neutralize a high percentage of the many types of HIV-1 in circulation. In some embodiments, broadly neutralizing antibodies to HIV-1 are distinct from other antibodies to HIV-1 in that they neutralize a high percentage (such as at least 90%, at least 95%, at least 96%, at least 97%, at least 98%, at least 99%) of the many types of HIV-1 in circulation.

According to a second aspect of the invention, the present invention provides a method of treating HIV in a human in need thereof comprising co-administering to a human a therapeutically effective amount of cabotegravir or a pharmaceutically acceptable salt thereof and a therapeutically effective amount of a gp120 binding protein the neutralizes HIV-1. The cabotegravir is as described above in the first aspect. The gp120 binding protein is as described above in the first aspect.

In an embodiment of the invention, the method provides the gp120 binding protein comprises a monoclonal antibody or fragment thereof.

In an embodiment of the invention, the method provides the gp120 comprises a heavy chain variable region (VH) comprising a heavy chain complementarity determining region (CDRH) having a CDRH1 amino acid sequence that comprises a sequence that is at least 80%, 85%, 90%, 95%, 98% or 99% identical to SEQ ID NO: 15, a CDRH2 amino acid sequence that comprises a sequence that is at least 80%, 85%, 90%, 95%, 98% or 99% identical to SEQ ID NO: 16, and a CDRH3 amino acid sequence that comprises a sequence that is at least 80%, 85%, 90%, 95%, 98% or 99% identical to SEQ ID NO: 17, and a light chain variable region (VL) comprising a light chain complementarity determining region (CDRL) having a CDRL1 amino acid that comprises a sequence that is at least 80%, 85%, 90%, 95%, 98% or 99% identical to SEQ ID NO: 18, a CDRL2 amino acid sequence that comprises a sequence that is at least 80%, 85%, 90%, 95%, 98% or 99% identical to SEQ ID NO: 19, and a CDRHL 3 amino acid sequence that comprises a sequence that is at least 80%, 85%, 90%, 95%, 98% or 99% identical to SEQ ID NO: 20.

In an embodiment of the invention, the method provides the gp120 binding protein comprises a VH comprising a heavy chain complementarity determining region (CDRH) having the CDRH1 amino acid sequence shown in in SEQ ID NO: 15, the CDRH2 amino acid sequence shown in in SEQ ID NO: 16, and the CDRH3 amino acid sequence shown in in SEQ ID NO: 17, and a VL comprising a light chain complementarity determining region (CDRL) having the CDRL1 amino acid sequence shown in in SEQ ID NO: 18, the CDRHL amino acid sequence shown in in SEQ ID NO: 19, and the CDRHL 3 amino acid sequence shown in in SEQ ID NO: 20.

In an embodiment of the invention, the method provides the gp120 binding protein comprises a variable heavy chain region amino acid sequence that comprises a sequence that is at least 80%, 85%, 90%, 95%, 98%, or 99% identical to SEQ ID NO: 1. In an embodiment of the invention, the method provides the gp120 binding protein comprises a variable heavy chain region amino acid sequence as shown in SEQ ID NO: 1.

In an embodiment of the invention, the method provides the gp120 binding protein comprises a variable light chain region amino acid sequence that comprises a sequence that is at least 80%, 85%, 90%, 95%, 98%, or 99% identical to SEQ ID NO: 2. In an embodiment of the invention, the method provides the gp120 binding protein comprises a variable light chain region amino acid sequence shown in SEQ ID NO: 2.

In an embodiment of the invention, the method provides the gp120 binding protein comprises a variable heavy chain region amino acid sequence that comprises a sequence that is at least 80%, 85%, 90%, 95%, 98%, or 99% identical to SEQ ID NO: 1 and a sequence that is at least 80%, 85%, 90%, 95%, 98%, or 99% identical to SEQ ID NO: 2.

In an embodiment of the invention, the method provides the gp120 binding protein comprises a variable heavy chain region amino acid sequence as shown in SEQ ID NO: 1 and a gp120 binding protein comprises a variable light chain region amino acid sequence shown in SEQ ID NO: 2.

In an embodiment of the invention, the method provides the gp120 binding protein comprises an antigen binding fragment. In an embodiment of the invention, the method provides the antigen binding is a Fv, Fab, F(ab′)₂, scFV or a scFV₂ fragment.

In an embodiment of the invention, the method provides the monoclonal antibody comprises a recombinant constant domain comprising a modification that increases binding to a neonatal Fc receptor relative to an unmodified constant domain, wherein the recombinant domain is an IgG1 constant domain comprising M428L and N434S mutations.

In an embodiment of the invention, the method provides for the co-administration of cabotegravir or a pharmaceutically acceptable salt thereof and a gp120 binding protein. In an embodiment of the invention, the method provides co-administration of the cabotegravir and the gp120 binding protein is sequential. In an embodiment of the invention, the method provides co-administration of the cabotegravir and the gp120 binding protein is simultaneous. In an embodiment of the invention, the co-administration is not simultaneous. The compounds may be administered at different schedules but could be administered within a given treatment cycle. In an embodiment of the invention, the co-administration of the compounds occurs within seconds, minutes, hours, days, weeks or months of each other. In an embodiment, co-administration occurs by any suitable means. In an embodiment of the invention, the method comprises administering the cabotegravir and the gp120 binding protein parenterally. The cabotegravir and the gp120 binding protein parenterally may be administered in the form of injectables. In an embodiment of the invention, each injectable may independently be administered intramuscularly. In an embodiment of the invention, each injectable may independently be administered subcutaneously. In an embodiment of the invention, the method comprises co-administering the cabotegravir and the gp120 binding protein wherein each injectable may independently be administered intravenously.

In an embodiment of the invention, the method comprises administering the cabotegravir and the gp120 binding protein to the human once every month, once every 2 months, once every 3 months, or once every 6 months.

According to a third aspect of the invention, there is provided a combination comprising cabotegravir or a pharmaceutically acceptable salt thereof and a gp120 binding protein, as described above, wherein the gp120 binding protein neutralizes HIV-1 for the use in treatment of HIV.

In an embodiment of the invention, the combination for use provides the gp120 binding protein comprises a monoclonal antibody or fragment thereof.

In an embodiment of the invention, the combination for use provides the gp120 binding protein comprises a heavy chain variable region (VH) comprising a heavy chain complementarity determining region (CDRH) having a CDRH1 amino acid sequence that comprises a sequence that is at least 80%, 85%, 90%, 95%, 98% or 99% identical to SEQ ID NO: 15, a CDRH2 amino acid sequence that comprises a sequence that is at least 80%, 85%, 90%, 95%, 98% or 99% identical to SEQ ID NO: 16, and a CDRH3 amino acid sequence that comprises a sequence that is at least 80%, 85%, 90%, 95%, 98% or 99% identical to SEQ ID NO: 17, and a light chain variable region (VL) comprising a light chain complementarity determining region (CDRL) having a CDRL1 amino acid that comprises a sequence that is at least 80%, 85%, 90%, 95%, 98% or 99% identical to SEQ ID NO: 18, a CDRL2 amino acid sequence that comprises a sequence that is at least 80%, 85%, 90%, 95%, 98% or 99% identical to SEQ ID NO: 19, and a CDRHL 3 amino acid sequence that comprises a sequence that is at least 80%, 85%, 90%, 95%, 98% or 99% identical to SEQ ID NO: 20.

In an embodiment of the invention, the combination for use provides the gp120 binding protein comprises a VH comprising a heavy chain complementarity determining region (CDRH) having the CDRH1 amino acid sequence shown in in SEQ ID NO: 15, the CDRH2 amino acid sequence shown in in SEQ ID NO: 16, and the CDRH3 amino acid sequence shown in in SEQ ID NO: 17, and a VL comprising a light chain complementarity determining region (CDRL) having the CDRL1 amino acid sequence shown in in SEQ ID NO: 18, the CDRHL amino acid sequence shown in in SEQ ID NO: 19, and the CDRHL 3 amino acid sequence shown in in SEQ ID NO: 20.

In an embodiment of the invention, the combination for use provides the gp120 binding protein comprises a variable heavy chain region amino acid sequence that comprises a sequence that is at least 80%, 85%, 90%, 95%, 98%, or 99% identical to SEQ ID NO: 1. In an embodiment of the invention, the combination for use provides the gp120 binding protein comprises a variable heavy chain region amino acid sequence as shown in SEQ ID NO: 1.

In an embodiment of the invention, the combination for use provides the gp120 binding protein comprises a variable light chain region amino acid sequence that comprises a sequence that is at least 80%, 85%, 90%, 95%, 98%, or 99% identical to SEQ ID NO: 2. In an embodiment of the invention, the combination for use provides the gp120 binding protein comprises a variable light chain region amino acid sequence shown in SEQ ID NO: 2.

In an embodiment of the invention, the combination for use provides the gp120 binding protein comprises an antigen binding fragment. In an embodiment of the invention, the combination for use provides the antigen binding is a Fv, Fab, F(ab′)₂, scFV or a scFV₂ fragment.

In an embodiment of the invention, the combination for use provides the monoclonal antibody comprises a recombinant constant domain comprising a modification that increases binding to a neonatal Fc receptor relative to an unmodified constant domain, wherein the recombinant domain is an IgG1 constant domain comprising M428L and N434S mutations.

In an embodiment of the invention, the combination for use provides the combination is co-administrated sequentially. In an embodiment of the invention, the combination for use provides the combination is co-administrated simultaneously. In an embodiment of the invention, the combination for use provides the combination is co-administrated parenterally.

In an embodiment of the invention, the combination for use provides the combination is co-administrated to the human once every month, once every 2 months, once every 3 months, or once every 6 months.

According to a further aspect of the invention, the invention provides use of cabotegravir or a pharmaceutically acceptable salt thereof and a gp120 binding protein as defined herein, in the manufacture or a medicament for use in the treatment of HIV.

According to a further aspect of the invention, the present invention provides a kit, wherein the kit comprises cabotegravir or a pharmaceutically acceptable salt thereof and a gp120 binding protein. In one embodiment, the kit comprises an integrase strand transfer inhibitor as disclosed herein and a gp120 binding protein as described herein. In one embodiment the kit comprises cabotegravir or a pharmaceutically acceptable salt thereof and a gp120 binding protein comprises a VH comprising a heavy chain complementarity determining region (CDRH) having the CDRH1 amino acid sequence shown in in SEQ ID NO: 15, the CDRH2 amino acid sequence shown in in SEQ ID NO: 16, and the CDRH3 amino acid sequence shown in in SEQ ID NO: 17, and a VL comprising a light chain complementarity determining region (CDRL) having the CDRL1 amino acid sequence shown in in SEQ ID NO: 18, the CDRHL amino acid sequence shown in in SEQ ID NO: 19, and the CDRHL 3 amino acid sequence shown in in SEQ ID NO: 20. In one embodiment the kit comprises cabotegravir or a pharmaceutically acceptable salt thereof and a gp120 binding protein comprises a VH comprising a heavy chain complementarity determining region (CDRH) having the CDRH1 amino acid sequence shown in in SEQ ID NO: 15, the CDRH2 amino acid sequence shown in in SEQ ID NO: 16, and the CDRH3 amino acid sequence shown in in SEQ ID NO: 17, and a VL comprising a light chain complementarity determining region (CDRL) having the CDRL1 amino acid sequence shown in in SEQ ID NO: 18, the CDRHL amino acid sequence shown in in SEQ ID NO: 19, and the CDRHL 3 amino acid sequence shown in in SEQ ID NO: 20, and an IgG1 constant domain comprising M428L and N434S mutations. In one embodiment, the kit comprises a syringe comprising the pharmaceutical composition of the invention as well as a leaflet comprising use instructions.

While not wishing to be bound by theory, it is thought that administering cabotegravir or a pharmaceutically acceptable salt thereof and a gp120 binding protein will target and disrupt two different parts of the HIV life cycle. In addition, it is believed that administering a compound with a high genetic barrier to resistance, the cabotegravir, with a compound with a low genetic barrier to resistance, the gp120 binding molecule, will reduce loss of therapeutic activity resulting from viral genetic code mutations.

The following non-limiting Examples illustrate the present invention.

### EXAMPLES

### Example 1: Preparation of Cabotegravir

TABLE 3
Composition of cabotegravir Injectable Suspension

| Component | Quantity (mg/mL) | Function |
|---|---|---|
| Cabotegravir | 200.0 | Active |
| Mannitol | 45.0 | Tonicity agent |
| Polysorbate 20 | 20.0 | Wetting agent |
| Polyethylene Glycol 3350 | 20.0 | Stabilizer |
| Water for Injection | QS to 1.0 mL | Solvent |

Cabotegravir, mannitol, polysorbate 20, PEG 3350, and water for injection were compounded and milled using a wet bead mill. The resulting suspension was filled into 3 mL, USP Type I glass vials at a fill volume of 1.5 mL, the vials are stoppered and sealed.

### Example 2: 2-Drug Combination

### Anti-HIV Evaluation of N6LS in 2-Drug Combination

### Drug Preparation

Provided below is an evaluation of the anti-HIV-1 activity and cytotoxicity of N6LS in two drug combination studies with Cabotegravir. N6LS was produced using standard methods described in the art. Stock solutions were prepared as listed in Table 4 and were thawed at room temperature on each day of assay setup to generate fresh working drug dilutions that were used that day in the assays. Working dilutions were not stored for re-use in subsequent experiments performed on different days. For each combination assay, N6LS was evaluated using a 20 nM high-test concentration with seven additional serial half-log dilutions. Each dilution of N6LS was tested in combination with five dilutions of the anti-HIV drug, Cabotegravir. In all cases the final DMSO concentration was <0.25%, which has been previously shown to have no effect in the described assays.

TABLE 4
Compound Solubilization Information

| | | | DMSO | Stock |
|---|---|---|---|---|
| Compound | Amount | Molecular | Volume | Concentration |
| ID | (mg) | Weight | (μL) | (mM) |
| N6LS | 5 | 150000 | 48 | 104 mg/ml |
| | | | | (0.693 mM) |
| Cabotegravir | 6.9 | 405.35 | 1,702.2 | 10 |

### Efficacy Evaluation in MT-4 Cells

For cell preparation, MT-4 cells (obtained from the NIH AIDS Research and Reference Reagent Program) were passaged in T-75 flasks prior to use in the antiviral assay. On the day preceding the assay, the cells were split 1:2 to assure they were in an exponential growth phase at the time of infection. Total cell and viability quantification were performed using a hemacytometer and trypan blue exclusion. Cell viability needed to be greater than 95% for the cells to be utilized in the assay. The cells were re-suspended in tissue culture medium and added to the drug-containing microtiter plates in a volume of 110 μL and at a seeding density of 5.0×103 cells/well.

For virus preparation, the virus used for these tests was the CXCR4-tropic virus strain HIV-1_{NL4-3}. This virus was obtained from the NIH AIDS Research and Reference Reagent Program and was grown in MT-4 cells for the production of stock virus pools. For each assay, a pre-titered aliquot of virus was removed from the freezer (−80° C.) and allowed to thaw slowly to room temperature in a biological safety cabinet. The virus was re-suspended and diluted into tissue culture medium such that the amount of virus added to each well, in a volume of 50 μL, was the amount determined to give between 85 to 95% cell-killing at 6 days post infection. TCID₅₀ calculations by endpoint titration in MT-4 cells indicated that the multiplicity of infection of these assays was approximately 0.01.

For Plate Format, a checkerboard plate format was used to test five concentrations of Cabotegravir with eight concentrations of N6LS. Combination antiviral efficacy was evaluated on three identical assay plates (i.e., triplicate measurements) that included cell control wells (cells only) and virus control wells (cells plus virus). Combination cytotoxicity was evaluated in parallel on two identical assay plates (i.e., duplicate measurements) that included cell control wells. Antiviral efficacy and cellular toxicity were monitored by MTS staining at the experimental endpoint.

For MTS Endpoint for cell viability, at assay termination, the soluble tetrazolium-based dye MTS (CellTiter 96 Reagent, Promega) was added to each well to determine cell viability and quantify compound toxicity. MTS is metabolized by the mitochondria enzymes of metabolically active cells to yield a soluble formazan product, allowing the rapid quantitative analysis of cell viability and compound cytotoxicity. The reagent is a stable, single solution that does not require preparation before use. At termination of the assay, 20 μL of MTS reagent was added per well and the microtiter plates were then incubated for 3-4 hours at 37° C., 5% CO₂ for the HIV cytoprotection assay; the incubation intervals were chosen based on empirically determined times for optimal MTS reduction. Adhesive plate sealers were used in place of the lids, the sealed plates were inverted several times to mix the soluble formazan product and the plates were read spectrophotometrically at 490/650 nm with a Molecular Devices SpectraMax i3 plate reader.

### Data Analysis

Combination antiviral assays were performed with MT-4 cells utilizing HIV-1_{NL4-3}, as described above. For each combination assay, five concentrations of Cabotegravir were tested with eight concentrations of N6LS. Three replicates were used to determine combination antiviral efficacy, and two replicates were used to determine combination cytotoxicity in uninfected MT-4. Each combination assay was performed three times.

The drug combination assay data was then analysed according to the method of Prichard and Shipman (Antiviral Research 14:181-206 [1990]) using the MacSynergy II program for data analysis and statistical evaluation. Briefly, the MacSynergy II program calculates the theoretical additive interactions of the drugs based on the Bliss Independence mathematical definition of expected effects for drug-drug interactions. The Bliss Independence model is based on statistical probability and assumes that the drugs act independently to affect virus replication; this Independent Effects model is also referred to as a Dual-Site (DS) model and is used for all combination analyses reported herein.

Theoretical additive interactions were calculated from the dose response curves for each drug used individually. This calculated additive surface, which represents predicted or additive interactions, was then subtracted from the experimentally determined dose-response surface to reveal regions of non-additive activity. The resulting surface would appear as a horizontal plane at 0% inhibition above calculated if the interactions were merely additive. Any peaks above this plane-of-additivity would be indicative of synergy. Similarly, any depressions below the plane-of-additivity would indicate antagonism. The 95% confidence intervals around the experimental dose-response surface were used to evaluate the data statistically and the volume of the peaks/depressions was calculated and used to quantify the volume of synergy/antagonism produced. The volume of the peaks observed in the synergy plots (in units of concentration times percent; e.g. μM²%, nM²%, nMμM %, etc.) was calculated by the program. This peak volume is the three-dimensional counterpart of the area under a 3-dimensional dose-response surface and is a quantitative measure of synergy or antagonism. For these studies, synergy is defined as drug combination yielding synergy volumes greater than 50. Slightly synergistic activity and highly synergistic activity have been operationally defined as yielding synergy volumes of 50-100 and >100, respectively. Additive drug interactions have synergy volumes in the range of −50 to 50, while synergy volumes between −50 and −100 are considered slightly antagonistic and those <−100 are highly antagonistic.

### Results

Table 5 summarizes the results from the antiviral testing of N6LS against HIV-1_{NL4-3} in MT-4. As summarized in Table 5 the combination of N6LS with the Cabotegravir for this study resulted in mainly synergistic or additive interactions.

TABLE 5
Antiviral Efficacy Results of N6LS in Combination with
Cabotegravir in MT-4 Cells (95% Confidence Values)

| | | | | Mean Synergy/ |
|---|---|---|---|---|
| | Synergy/Antagonism Volume | | | Antagonism |
| | (nM² %)¹ | | | Volume |
| Compound¹ | Result 1 | Result 2 | Result 3 | (nM²; n = 3)² |

Integrase strand transfer inhibitor

| | | | | |
|---|---|---|---|---|
| Cabotegravir | 2.32/−45.27 | 187.5/0 | 61.7/−39.56 | 83.84/−28.28 |

¹The MacSynergy II program takes the raw data from individual experiments and calculates a positive (synergy) or negative (antagonism) value for each drug-drug combination [these values can be found in the Antiviral Synergy Plot (95%) sections of the spreadsheets found in Appendices II-IX]. Positive values are summed to give a Volume of Synergy and negative values are summed to give a Volume of Antagonism (both values are reported for each experiment).
²The Antiviral Synergy Plot (95%) datasets from multiple experiments (n = 3) are combined and arithmetic means are calculated for each drug-drug concentration. The positive and negative values are individually summed to respectively give Mean Volumes for synergistic and antagonistic interactions.

### SEQUENCE LISTINGS

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

TABLE 6
N6 Sequence Listings

| | | |
|---|---|---|
| SEQ ID NO: 1 | RAHLVQSGTAMKKPGASVRVSCQTSGYTFTAHIL | an amino acid |
| | FWFRQAPGRGLEWVGWIKPQYGAVNFGGGFRDR | sequence of the VH |
| | VTLTRDVYREIAYMDIRGLKPDDTAVYYCARDRSY | of the N6 mAb |
| | GDSSWALDAWGQGTTVVVSA | |
| SEQ ID NO: 2 | YIHVTQSPSSLSVSIGDRVTINCQTSQGVGSDLH | an amino acid |
| | WYQHKPGRAPKLLIHHTSSVEDGVPSRFSGSGFH | sequence of the VL |
| | TSFNLTISDLQADDIATYYCQVLQFFGRGSRLHIK | of the N6 mAb |
| SEQ ID NO: 3 | CGAGCGCACCTGGTACAATCAGGGACTGCGATG | a nucleic acid |
| | AAGAAACCGGGGGCCTCAGTAAGAGTCTCCTGC | sequence encoding |
| | CAGACCTCTGGATACACCTTTACCGCCCACATAT | the VH of the N6 |
| | TATTTTGGTTCCGACAGGCCCCCGGGCGAGGAC | mAb |
| | TTGAGTGGGTGGGGTGGATCAAGCCACAATATG | |
| | GGGCCGTGAATTTTGGTGGTGGTTTTCGGGACA | |
| | GGGTCACATTGACTCGAGACGTATATAGAGAGA | |
| | TTGCGTACATGGACATCAGAGGCCTTAAACCTGA | |
| | CGACACGGCCGTCTATTACTGTGCGAGAGACCG | |
| | TTCCTATGGCGACTCCTCTTGGGCCTTAGATGCC | |
| | TGGGGACAGGGAACGACGGTCGTCGTCTCCGCG | |
| SEQ ID NO: 4 | TACATCCACGTGACCCAGTCTCCGTCCTCCCTGT | a nucleic acid |
| | CTGTGTCTATTGGAGACAGAGTCACCATCAATTG | sequence encoding |
| | CCAGACGAGTCAGGGTGTTGGCAGTGACCTACA | the VL of the N6 mAb |
| | TTGGTATCAACACAAACCGGGGAGAGCCCCTAA | |
| | ACTCTTGATCCACCATACCTCTTCTGTGGAAGAC | |
| | GGTGTCCCCTCAAGATTCAGCGGCTCTGGATTT | |
| | CACACATCTTTTAATCTGACCATCAGCGACCTAC | |
| | AGGCTGACGACATTGCCACATATTACTGTCAAGT | |
| | TTTACAATTTTTCGGCCGAGGGAGTCGACTC | |
| | CATATTAAA | |
| SEQ ID NO: 5 | RAHLVQSGTAMKKPGASVRVSCQTSGYTFTAHIL | a heavy chain |
| | FWFRQAPGRGLEWVGWIKPQYGAVNFGGGFRDR | sequence including |
| | VTLTRDVYREIAYMDIRGLKPDDTAVYYCARDRSY | the N6 VH |
| | GDSSWALDAWGQGTTVVVSAASTKGPSVFPLAPS | |
| | SKSTSGGTAALGCLVKDYFPEPVTVSWNSGALTS | |
| | GVHTFPAVLQSSGLYSLSSVVTVPSSSLGTQTYIC | |
| | NVNHKPSNTKVDKKVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVVS | |
| | VLTVLHQDWLNGKEYKCKVSNKALPAPIEKTISKA | |
| | KGQPREPQVYTLPPSRDELTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | LSPGK | |
| SEQ ID NO: 6 | CGAGCGCACCTGGTACAATCAGGGACTGCGATG | a nucleic acid |
| | AAGAAACCGGGGGCCTCAGTAAGAGTCTCCTGC | sequence encoding a |
| | CAGACCTCTGGATACACCTTTACCGCCCACATAT | heavy chain including |
| | TATTTTGGTTCCGACAGGCCCCCGGGCGAGGAC | the N6 VH |
| | TTGAGTGGGTGGGGTGGATCAAGCCACAATATG | |
| | GGGCCGTGAATTTTGGTGGTGGTTTTCGGGACA | |
| | GGGTCACATTGACTCGAGACGTATATAGAGAGA | |
| | TTGCGTACATGGACATCAGAGGCCTTAAACCTGA | |
| | CGACACGGCCGTCTATTACTGTGCGAGAGACCG | |
| | TTCCTATGGCGACTCCTCTTGGGCCTTAGATGCC | |
| | TGGGGACAGGGAACGACGGTCGTCGTCTCCGCG | |
| | GCGTCGACCAAGGGCCCATCGGTCTTCCCCCTG | |
| | GCACCCTCCTCCAAGAGCACCTCTGGGGGCACA | |
| | GCGGCCCTGGGCTGCCTGGTCAAGGACTACTTC | |
| | CCCGAACCGGTGACGGTGTCGTGGAACTCAGGC | |
| | GCCCTGACCAGCGGCGTGCACACCTTCCCGGCT | |
| | GTCCTACAGTCCTCAGGACTCTACTCCCTCAGCA | |
| | GCGTGGTGACCGTGCCCTCCAGCAGCTTGGGCA | |
| | CCCAGACCTACATCTGCAACGTGAATCACAAGCC | |
| | CAGCAACACCAAGGTGGACAAGAAAGTTGAGCC | |
| | CAAATCTTGTGACAAAACTCACACATGCCCACCG | |
| | TGCCCAGCACCTGAACTCCTGGGGGGACCGTCA | |
| | GTCTTCCTCTTCCCCCCAAAACCCAAGGACACCC | |
| | TCATGATCTCCCGGACCCCTGAGGTCACATGCGT | |
| | GGTGGTGGACGTGAGCCACGAAGACCCTGAGGT | |
| | CAAGTTCAACTGGTACGTGGACGGCGTGGAGGT | |
| | GCATAATGCCAAGACAAAGCCGCGGGAGGAGCA | |
| | GTACAACAGCACGTACCGTGTGGTCAGCGTCCT | |
| | CACCGTCCTGCACCAGGACTGGCTGAATGGCAA | |
| | GGAGTACAAGTGCAAGGTCTCCAACAAAGCCCT | |
| | CCCAGCCCCCATCGAGAAAACCATCTCCAAAGCC | |
| | AAAGGGCAGCCCCGAGAACCACAGGTGTACACC | |
| | CTGCCCCCATCCCGGGATGAGCTGACCAAGAAC | |
| | CAGGTCAGCCTGACCTGCCTGGTCAAAGGCTTC | |
| | TATCCCAGCGACATCGCCGTGGAGTGGGAGAGC | |
| | AATGGGCAGCCGGAGAACAACTACAAGACCACG | |
| | CCTCCCGTGCTGGACTCCGACGGCTCCTTCTTCC | |
| | TCTACAGCAAGCTCACCGTGGACAAGAGCAGGT | |
| | GGCAGCAGGGGAACGTCTTCTCATGCTCCGTGA | |
| | TGCATGAGGCTCTGCACAACCACTACACGCAGAA | |
| | GAGCCTCTCCCTGTCTCCG GGTAAA | |
| SEQ ID NO: 7 | YIHVTQSPSSLSVSIGDRVTINCQTSQGVGSDLH | a light chain |
| | WYQHKPGRAPKLLIHHTSSVEDGVPSRFSGSGFH | sequence including |
| | TSFNLTISDLQADDIATYYCQVLQFFGRGSRLHIK | the N6 VL |
| | RTVAAPSVFIFPPSDEQLKSGTASVVCLLNNFYPRE | |
| | AKVQWKVDNALQSGNSQESVTEQDSKDSTYSLS | |
| | STLTLSKADYEKHKVYACEVTHQGLSSP | |
| | VTKSFNRGEC | |
| SEQ ID NO: 8 | TACATCCACGTGACCCAGTCTCCGTCCTCCCTGT | a nucleic acid |
| | CTGTGTCTATTGGAGACAGAGTCACCATCAATTG | sequence encoding a |
| | CCAGACGAGTCAGGGTGTTGGCAGTGACCTACA | light chain including |
| | TTGGTATCAACACAAACCGGGGAGAGCCCCTAA | the N6 VH |
| | ACTCTTGATCCACCATACCTCTTCTGTGGAAGAC | |
| | GGTGTCCCCTCAAGATTCAGCGGCTCTGGATTT | |
| | CACACATCTTTTAATCTGACCATCAGCGACCTAC | |
| | AGGCTGACGACATTGCCACATATTACTGTCAAGT | |
| | TTTACAATTTTTCGGCCGAGGGAGTCGACTCCAT | |
| | ATTAAACGTACGGTGGCTGCACCATCTGTCTTCA | |
| | TCTTCCCGCCATCTGATGAGCAGTTGAAATCTGG | |
| | AACTGCCTCTGTTGTGTGCCTGCTGAATAACTTC | |
| | TACCCCAGAGAAGCCAAAGTGCAGTGGAAGGTG | |
| | GACAACGCCCTGCAGAGCGGAAACAGCCAGGAA | |
| | AGCGTGACAGAGCAGGATTCCAAGGATTCCACA | |
| | TACAGCCTGAGCAGCACACTGACACTGTCCAAG | |
| | GCCGACTACGAGAAGCACAAGGTGTACGCCTGC | |
| | GAAGTGACACACCAGGGACTGTCCTCCCCTGTG | |
| | ACAAAGAGCTTCAACAGAGGAGAATGC | |
| SEQ ID NO: 9 | RAHLVQSGTAMKKPGASVRVSCQTSGYTFTAHIL | a heavy chain |
| | FWFRQAPGRGLEWVGWIKPQYGAVNFGGGFRDR | sequence including |
| | VTLTRDVYREIAYMDIRGLKPDDTAVYYCARDRSY | the N6 VH as isolated |
| | GDSSWALDAWGQGTTVVVSAASTKGPSVFPLAPS | from a human donor, |
| | SKSTSGGTAALGCLVKDYFPEPVTVSWNSGALTS | which includes |
| | GVHTFPAVLQSSGLYSLSSVVTVPSSSLGTQTYIC | polymorphisms |
| | NVNHKPSNTKVDKRVEPKSCDKTHTCPPCPAPELL | compared to SEQ ID |
| | GGPSVFLFPPKPKDTLMISRTPEVTCVVVDVSHED | NO: 5 |
| | PEVKFNWYVDGVEVHNAKTKPREEQYNSTYRVVS | |
| | VLTVLHQDWLNGEEYKCKVSNKALPAPIEKTISKA | |
| | KGQPREPQVYTLPPSREEMTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | LSP GK | |
| SEQ ID NO: 10 | CGAGCGCACCTGGTACAATCAGGGACTGCGATG | a sequence of the |
| | AAGAAACCGGGGGCCTCAGTAAGAGTCTCCTGC | nucleic acid molecule |
| | CAGACCTCTGGATACACCTTTACCGCCCACATAT | encoding the N6 VH |
| | TATTTTGGTTCCGACAGGCCCCCGGGCGAGGAC | as isolated from a |
| | TTGAGTGGGTGGGGTGGATCAAGCCACAATATG | human donor |
| | GGGCCGTGAATTTTGGTGGTGGTTTTCGGGACA | |
| | GGGTCACATTGACTCGAGACGTATATAGAGAGA | |
| | TTGCGTACATGGACATCAGAGGCCTTAAACCTGA | |
| | CGACACGGCCGTCTATTACTGTGCGAGAGACCG | |
| | TTCCTATGGCGACTCCTCTTGGGCCTTAGATGCC | |
| | TGGGGACAGGGAACGACGGTCGTCGTCTCCGCG | |
| | GCCTCCACCAAGGGCCCATCGGTCTTCCCCCTG | |
| | GCACCCTCCTCCAAGAGCACCTCTGGGGGCACA | |
| | GCGGCCCTGGGCTGCCTGGTCAAGGACTACTTC | |
| | CCCGAACCGGTGACGGTGTCGTGGAACTCAGGC | |
| | GCCCTGACCAGCGGCGTGCACACCTTCCCGGCT | |
| | GTCCTACAGTCCTCAGGACTCTACTCCCTCAGCA | |
| | GCGTGGTGACCGTGCCCTCCAGCAGCTTGGGCA | |
| | CCCAGACCTACATCTGCAACGTGAATCACAAGCC | |
| | CAGCAACACCAAGGTGGACAAGAGAGTTGAGCC | |
| | CAAATCTTGTGACAAAACTCACACATGCCCACCG | |
| | TGCCCAGCACCTGAACTCCTGGGGGGACCGTCA | |
| | GTCTTCCTCTTCCCCCCAAAACCCAAGGACACCC | |
| | TCATGATCTCCCGGACCCCTGAGGTCACATGCGT | |
| | GGTGGTGGACGTGAGCCACGAAGACCCTGAGGT | |
| | CAAGTTCAACTGGTACGTGGACGGCGTGGAGGT | |
| | GCATAATGCCAAGACAAAGCCGCGGGAGGAGCA | |
| | GTACAACAGCACGTACCGTGTGGTCAGCGTCCT | |
| | CACCGTCCTGCACCAGGACTGGCTGAATGGCGA | |
| | GGAGTACAAGTGCAAGGTCTCCAACAAAGCCCT | |
| | CCCAGCCCCCATCGAGAAAACCATCTCCAAAGCC | |
| | AAAGGGCAGCCCCGAGAACCACAGGTGTACACC | |
| | CTGCCCCCATCCCGGGAGGAGATGACCAAGAAC | |
| | CAGGTCAGCCTGACCTGCCTTGTCAAAGGCTTCT | |
| | ATCCCAGCGACATCGCCGTGGAGTGGGAGAGCA | |
| | ATGGGCAGCCGGAGAACAACTACAAGACCACGC | |
| | CTCCCGTGCTGGACTCCGACGGCTCCTTCTTCCT | |
| | CTACAGCAAGCTCACCGTGGACAAGAGCAGGTG | |
| | GCAGCAGGGGAACGTCTTCTCATGCTCCGTGAT | |
| | GCATGAGGCTCTGCACAACCACTACACGCAGAA | |
| | GAGCCTCTCCCTGTCTCCG GGTAAA | |
| SEQ ID NO: 11 | CGAGGGCACTTGGTGCAGTCAGGGACTGAGGTG | a nucleotide |
| | AAGAAACCGGGGGCCTCAGTGAGAGTCTCCTGC | sequence encoding |
| | GAGACTTCTGGATACACCTTCACCGCCTACATTT | the N6 I1 VH |
| | TACATTGGTTCCGACAGGCCCCCGGACGAGGGC | intermediate |
| | TTGAGTGGATGGGGTGGATCAAGCCAAAATATG | |
| | GAGCCGTCAATTATGCTCATGCATTTCAGGGCA | |
| | GGGTCACCCTGACCAGAGACATATATAGAGACA | |
| | CTGCATACATGGACTTGAGTGGCCTAAGATTCG | |
| | ACGACACGGCCGTCTATTACTGTGCGAGAGATC | |
| | GCGTTTATGACGATTCGTCTTGGCAATTGGATCC | |
| | CTGGGGCCAGGGAACTTCGGTCATCGTCTCCTC | |
| | A | |
| SEQ ID NO: 12 | CGAGGGCACTTGGTGCAGTCAGGGACTGAGGTG | a nucleotide |
| | AAGAAACCGGGGGCCTCAGTGAGAGTCTCCTGC | sequence encoding |
| | GAGACTTCTGGATACACCTTCACCGCCCACATTT | the N6 I2 VH |
| | TACATTGGTTCCGACAGGCCCCCGGACGAGGGC | intermediate |
| | TTGAGTGGATGGGGTGGATCAAGCCAAAATATG | |
| | GAGCCGTCAATTATGCTCATGCATTTCAGGGCA | |
| | GGGTCACCCTGACCAGAGACATATATAGAGACA | |
| | CTGCATACATGGACTTGAGTGGCCTAAGATTCG | |
| | ACGACACGGCCGTCTATTACTGTGCGAGAGATC | |
| | GCGTTTATGACGATTCGTCTTGGCAATTGGATCC | |
| | CTGGGGCCAGGGAACTTCGGTCATCGTCTCCTC | |
| | A | |
| SEQ ID NO: 13 | CGAGCGCACTTGGTGCAGTCAGGGACTGCGGTG | a nucleotide |
| | AAGAAACCGGGGGCCTCAGTGAGAGTCTCCTGC | sequence encoding |
| | GAGACTTCTGGATACACCTTCACCGCCCACATTT | the N6 I3 VH |
| | TATATTGGTTCCGACAGGCCCCCGGACGAGGGC | intermediate |
| | TTGAGTGGGTGGGGTGGATCAAGCCACAATATG | |
| | GGGCCGTGAATTTTGGTGGTGGTTTTCGGGGCA | |
| | GGGTCACCCTGACCAGAGACATATATAGAGACA | |
| | CTGCATACATGGACATCAGTGGCCTAAGATTCGA | |
| | CGACACGGCCGTCTATTACTGTGCGAGAGATCG | |
| | CTCCTATGACGACTCCTCTTGGGCCTTAGATGCC | |
| | TGGGGACAGGGAACGACGGTCGTCGTCTCCGCG | |
| SEQ ID NO: 14 | CGAGCGCACCTGGTACAATCAGGGACTGCGATG | a nucleotide |
| | AAGAAACCGGGGGCCTCAGTAAGAGTCTCCTGC | sequence encoding |
| | CAGACCTCTGGATACACCTTTACCGCCCACATAT | the N6 I4 VH |
| | TATTTTGGTTCCGACAGGCCCCCGGGCGAGGAC | intermediate |
| | TTGAGTGGGTGGGGTGGATCAAGCCACAATATG | |
| | GGGCCGTGAATTTTGGTGGTGGTTTTCGGGACA | |
| | GGGTCACATTGACTCGAGACATATATAGAGAGA | |
| | TTGCGTACATGGACATCAGAGGCCTTAAACTTGA | |
| | CGACACGGCCGTCTATTACTGTGCGAGAGACCG | |
| | TTCCTATGGCGACTCCTCTTGGGCCTTAGATGCC | |
| | TGGGGACAGGGAACGACGGTCGTCGTCTCCGCG | |
| SEQ ID NO: 15 | AHILF | an amino acid |
| | | sequence of N6 |
| | | CDRH1 |
| SEQ ID NO: 16 | WIKPQYGAVNFGGGFRD | an amino acid |
| | | sequence of N6 |
| | | CDRH2 |
| SEQ ID NO: 17 | DRSYGDSSWALDA | an amino acid |
| | | sequence of N6 |
| | | CDRH3 |
| SEQ ID NO: 18 | QTSQGVGSDLH | an amino acid |
| | | sequence of N6 |
| | | CDRL1 |
| SEQ ID NO: 19 | HTSSVED | an amino acid |
| | | sequence of N6 |
| | | CDRL2 |
| SEQ ID NO: 20 | QVLQF | an amino acid |
| | | sequence of N6 |
| | | CDRL3 |
| SEQ ID NO: 21 | MGWSCIILFLVATATGVHSRAHLVQSGTAMKKPG | an amino acid |
| | ASVRVSCQTSGYTFTAHILFWFRQAPGRGLEWVG | sequence of the N6- |
| | WIKPQYGAVNFGGGFRDRVTLTRDVYREIAYMDI | LS heavy chain |
| | RGLKPDDTAVYYCARDRSYGDSSWALDAWGQGT | |
| | TVVVSAASTKGPSVFPLAPSSKSTSGGTAALGCLV | |
| | KDYFPEPVTVSWNSGALTSGVHTFPAVLQSSGLYS | |
| | LSSVVTVPSSSLGTQTYICNVNHKPSNTKVDKKVE | |
| | PKSCDKTHTCPPCPAPELLGGPSVFLFPPKPKDTL | |
| | MISRTPEVTCVVVDVSHEDPEVKFNWYVDGVEVH | |
| | NAKTKPREEQYNSTYRVVSVLTVLHQDWLNGKEY | |
| | KCKVSNKALPAPIEKTISKAKGQPREPQVYTLPPSR | |
| | DELTKNQVSLTCLVKGFYPSDIAVEWESNGQPEN | |
| | NYKTTPPVLDSDGSFFLYSKLTVDKSRWQQGNVF | |
| | SCSVLHEALHSHYTQKSLSLSPGK | |
| SEQ ID NO: 22 | MGWSCIILFLVATATGVHSYIHVTQSPSSLSVSIG | an amino acid |
| | DRVTINCQTSQGVGSDLHWYQHKPGRAPKLLIHH | sequence of the N6- |
| | TSSVEDGVPSRFSGSGFHTSFNLTISDLQADDIAT | LS light chain |
| | YYCQVLQFFGRGSRLHIKRTVAAPSVFIFPPSDEQL | |
| | KSGTASVVCLLNNFYPREAKVQWKVDNALQSGNS | |
| | QESVTEQDSKDSTYSLSSTLTLSKADYEKHKVYAC | |
| | EVTHQGLSSPVTKSFNRGEC | |

1.-44. (canceled)

45. A method of treating an HIV infection in a human in need thereof comprising:
administering to a human a therapeutically effective amount of cabotegravir or a pharmaceutically acceptable salt thereof and a therapeutically effective amount of an HIV gp120 binding protein or an antigen binding fragment thereof.

46. The method according to claim 45, wherein the HIV gp120 binding protein neutralizes HIV-1.

47. The method according to claim 45, wherein the HIV gp120 binding protein comprises a heavy chain variable region (VH) comprising a heavy chain complementarity determining region (HCDR)1, a HCDR2, and a HCDR3 set forth as SEQ ID NOs: 15, 16, and 17, respectively; a light chain variable region (V_{L}) comprising a light chain complementarity determining region (LCDR)1, a LCDR2, and a LCDR3 set forth as SEQ ID NOs: 18, 19, and 20, respectively; and a recombinant constant domain comprising one or more amino acid modifications that increase the half-life of the antibody.

48. The method according to claim 47, wherein the V_{H} comprises the amino acid sequence having at least 95% sequence identity to SEQ ID NO: 1, and wherein the V_{H} has the HCDR1 of SEQ ID NO: 15, the HCDR2 of SEQ ID NO: 16, and the HCDR3 of SEQ ID NO: 17.

49. The method according to claim 47, wherein the V_{H} comprises the amino acid sequence of SEQ ID NO: 1.

50. The method according to claim 47, wherein the V_{L} comprises the amino acid sequence having at least 95% sequence identity to SEQ ID NO: 2, and wherein the V_{L} has the LCDR1 of SEQ ID NO: 18, the LCDR2 of SEQ ID NO: 19, and the LCDR3 of SEQ ID NO: 20.

51. The method according to claim 47, wherein the V_{L} comprises the amino acid sequence of SEQ ID NO: 2.

52. The method according to claim 47, wherein the recombinant constant domain comprises one or more mutations from the group consisting of: M252Y, S254T, T256E, M428L, N434S, T250Q, M428L, V259I, V308F, M428L, N434A, T307A, E380A, N434A, H433K, N434F, and Y436H.

53. The method according to claim 47, wherein the recombinant constant domain comprises one or more mutations from the group consisting of: M252Y/S254T/T256E, M428L/N434S, T250Q/M428L, V259I/V308F/M428L, E380A/N434A, T307A/E380A/N434A, and H433K/N434F.

54. The method according to claim 47, wherein the recombinant constant domain comprises M428L and N434S mutations.

55. The method according to claim 47, wherein the HIV gp120 binding protein comprises an antigen binding fragment.

56. The method according to claim 55, wherein the antigen binding is a Fv, Fab, F(ab′)₂, scFV or a scFV₂ fragment.

57. The method according to claim 47, wherein the HIV gp120 binding protein is an isolated monoclonal antibody.

58. The method according to claim 57, wherein the antibody is an IgG, IgA or IgM antibody.

59. The method according to claim 47, wherein the cabotegravir or a pharmaceutically acceptable salt thereof and the HIV gp120 binding protein or an antigen binding fragment thereof are administered separately.

60. The method according to claim 47, the cabotegravir or a pharmaceutically acceptable salt thereof and the HIV gp120 binding protein or an antigen binding fragment thereof are administered simultaneously.

61. The method according to claim 47, wherein the cabotegravir or a pharmaceutically acceptable salt thereof and the HIV gp120 binding protein or an antigen binding fragment thereof are administered parenterally.

62. The method according to claim 47, wherein the cabotegravir or a pharmaceutically acceptable salt thereof and the HIV gp120 binding protein or an antigen binding fragment thereof are administered once every month, once every 2 months, once every 3 months, or once every 6 months.


SEQUENCE LISTING (22 sequences)

SEQ ID NO: 1 - protein, 122 aa, Artificial Sequence [sequence omitted]

SEQ ID NO: 2 - protein, 103 aa, Artificial Sequence [sequence omitted]

SEQ ID NO: 3 - DNA, 366 bp, Artificial Sequence
  A nucleic acid sequence encoding the VH of the N6 mAb
cgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcg

SEQ ID NO: 4 - DNA, 309 bp, Artificial Sequence
  A nucleic acid sequence encoding the VL of the N6 mAb
tacatccacgtgacccagtctccgtcctccctgtctgtgtctattggagacagagtcaccatcaattgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaatttttcggccgagggagtcgactccatattaaa

SEQ ID NO: 5 - protein, 452 aa, Artificial Sequence
  A heavy chain sequence including the N6 VH
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1356 bp, Artificial Sequence
  A nucleic acid sequence encoding a heavy chain including the N6 VH
cgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcggcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 210 aa, Artificial Sequence
  A light chain sequence including the N6 VL
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 630 bp, Artificial Sequence
  A nucleic acid sequence encoding a light chain including the N6 VH
tacatccacgtgacccagtctccgtcctccctgtctgtgtctattggagacagagtcaccatcaattgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaatttttcggccgagggagtcgactccatattaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgc

SEQ ID NO: 9 - protein, 452 aa, Artificial Sequence
  A heavy chain sequence including the N6 VH as isolated from a human donor, which include polymorphism compared to SEQ ID NO 5
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGEEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1356 bp, Artificial Sequence
  A sequence of the nucleic acid molecule encoding the N6 VH as isolated from a human donor
cgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcgaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgccttgtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - DNA, 366 bp, Artificial Sequence
  A nucleotide sequence encoding the N6 I1 VH intermediate
cgagggcacttggtgcagtcagggactgaggtgaagaaaccgggggcctcagtgagagtctcctgcgagacttctggatacaccttcaccgcctacattttacattggttccgacaggcccccggacgagggcttgagtggatggggtggatcaagccaaaatatggagccgtcaattatgctcatgcatttcagggcagggtcaccctgaccagagacatatatagagacactgcatacatggacttgagtggcctaagattcgacgacacggccgtctattactgtgcgagagatcgcgtttatgacgattcgtcttggcaattggatccctggggccagggaacttcggtcatcgtctcctca

SEQ ID NO: 12 - DNA, 366 bp, Artificial Sequence
  A nucleotide sequence encoding the N6 I2 VH intermediate
cgagggcacttggtgcagtcagggactgaggtgaagaaaccgggggcctcagtgagagtctcctgcgagacttctggatacaccttcaccgcccacattttacattggttccgacaggcccccggacgagggcttgagtggatggggtggatcaagccaaaatatggagccgtcaattatgctcatgcatttcagggcagggtcaccctgaccagagacatatatagagacactgcatacatggacttgagtggcctaagattcgacgacacggccgtctattactgtgcgagagatcgcgtttatgacgattcgtcttggcaattggatccctggggccagggaacttcggtcatcgtctcctca

SEQ ID NO: 13 - DNA, 366 bp, Artificial Sequence
  A nucleotide sequence encoding the N6 I3 VH intermediate.
cgagcgcacttggtgcagtcagggactgcggtgaagaaaccgggggcctcagtgagagtctcctgcgagacttctggatacaccttcaccgcccacattttatattggttccgacaggcccccggacgagggcttgagtgggtggggtggatcaagccacaatatggggccgtgaattttggtggtggttttcggggcagggtcaccctgaccagagacatatatagagacactgcatacatggacatcagtggcctaagattcgacgacacggccgtctattactgtgcgagagatcgctcctatgacgactcctcttgggccttagatgcctggggacagggaacgacggtcgtcgtctccgcg

SEQ ID NO: 14 - DNA, 366 bp, Artificial Sequence
  A nucleotide sequence encoding the N6 I4 VH intermediate.
cgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacatatatagagagattgcgtacatggacatcagaggccttaaacttgacgacacggccgtctattactgtgcgagagaccgttcctatggcgactcctcttgggccttagatgcctggggacagggaacgacggtcgtcgtctccgcg

SEQ ID NO: 15 - protein, 5 aa, Artificial Sequence
  An amino acid sequence of N6 CDRH1
AHILF

SEQ ID NO: 16 - protein, 17 aa, Artificial Sequence
  An amino acid sequence of N6 CDRH2
WIKPQYGAVNFGGGFRD

SEQ ID NO: 17 - protein, 13 aa, Artificial Sequence
  An amino acid sequence of N6 CDRH3
DRSYGDSSWALDA

SEQ ID NO: 18 - protein, 11 aa, Artificial Sequence
  An amino acid sequence of N6 CDRL1
QTSQGVGSDLH

SEQ ID NO: 19 - protein, 7 aa, Artificial Sequence
  An amino acid sequence of N6 CDRL2
HTSSVED

SEQ ID NO: 20 - protein, 5 aa, Artificial Sequence
  An amino acid sequence of N6 CDRL3
QVLQF

SEQ ID NO: 21 - protein, 471 aa, Artificial Sequence
  An amino acid sequence of the N6-LS heavy chain
MGWSCIILFLVATATGVHSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 22 - protein, 229 aa, Artificial Sequence
  An amino acid sequence of the N6-LS light chain
MGWSCIILFLVATATGVHSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC